SEQUENCE LISTING (17 sequences)

SEQ ID NO: 1 - protein, 10 aa, Homo sapiens
KSAPATGGVK

SEQ ID NO: 2 - protein, 7 aa, Homo sapiens
APATGGV

SEQ ID NO: 3 - protein, 22 aa, Homo sapiens
MRGMKLLGALLALAALLQGAVS

SEQ ID NO: 4 - protein, 20 aa, Homo sapiens
MYRMQLLSCIALSLALVTNS

SEQ ID NO: 5 - protein, 22 aa, Homo sapiens
MRYTGLMGTLLTLVNLLQLAGT

SEQ ID NO: 6 - protein, 15 aa, synthetic construct
GGGGSGGGGSGGGGS

SEQ ID NO: 7 - protein, 217 aa, synthetic construct
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 12 aa, synthetic construct
ESKYGPPCPSCP

SEQ ID NO: 9 - protein, 44 aa, synthetic construct
TTTPAPRPPTPAPTIASQPLSLRPEACRPAAGGAVHTRGLDFAC

SEQ ID NO: 10 - protein, 110 aa, synthetic construct
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 11 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 18 aa, synthetic construct
GSTSGSGKPGSGEGSTKG

SEQ ID NO: 13 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 14 - protein, 41 aa, synthetic construct
RSKRSRLLHSDYMNMTPRRPGPTRKHYQPYAPPRDFAAYRS

SEQ ID NO: 15 - protein, 36 aa, synthetic construct
RDQRLPPDAHKPPGGGSFRTPIQEEQADAHSTLAKI

SEQ ID NO: 16 - protein, 42 aa, synthetic construct
KRGRKKLLYIFKQPFMRPVQTTQEEDGCSCRFPEEEEGGCEL

SEQ ID NO: 17 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0170241 A1
(43) Pub. Date: May 29, 2025

(54) CHIMERIC ANTIGEN RECEPTOR T-CELL TREATMENTS TARGETED TO CHROMATIN FRAGMENTS AND EXTRACELLULAR TRAPS

(71) Applicants: BELGIAN VOLITION SRL, Isnes (BE); XENETIC BIOSCIENCES INC., Framingham, MA (US)

(72) Inventors: Mark Edward ECCLESTON, Isnes (BE); Dmitry Dmitrievich GENKIN, Framingham, MA (US)

(21) Appl. No.: 18/843,056

(22) PCT Filed: Mar. 1, 2023
(86) PCT No.: PCT/EP2023/055230

§ 371 Date: Aug. 30, 2024

(30) Foreign Application Priority Data
Mar. 1, 2022 (GB) 2202842.7

(51) Int. Cl. A61K 40/41; A61K 40/11; A61K 40/31; C07K 16/18

(52) U.S. Cl. CPC A61K 40/41; A61K 40/11; A61K 40/31; C07K 16/18; C07K 2317/24; C07K 2317/622

(57) ABSTRACT

The invention relates to engineered immune cells comprising a chimeric antigen receptor or a T cell receptor, wherein said CAR or TCR comprises an antigen binding domain capable of specific binding to one or more epitopes associated with neutrophil extracellular traps.

### FIELD OF THE INVENTION

The present invention relates to a cell expressing a Chimeric Antigen Receptor (CAR) or a T Cell Receptor (TCR) wherein the CAR or TCR is targeted to bind to an extracellular trap, especially a neutrophil extracellular trap (NET). The invention also relates to a cell expressing a CAR or TCR that binds to NETs and additionally expressing a deoxyribonuclease enzyme. Cells of the invention are useful for the treatment of conditions associated with inappropriate NETosis and especially for the treatment of cancer.

### BACKGROUND OF THE INVENTION

T cells (also known as a T lymphocytes) are derived from hematopoietic stem cells in the red bone marrow as immature T lymphocytes. The immature T lymphocytes migrate to the thymus, where they are known as thymocytes, for maturation during which they develop a functional TCR. The expressed TCR is generated from millions of possible combinations of variable (V), diversity (D), and joining (J) gene segments in the genes that code for the variable regions of the TCR. This produces a huge library of thymocytes containing different randomly generated variable regions with the potential to bind a vast number of different epitopes. Most thymocytes generated would be self-reactive because they include TCRs that would bind to epitopes present in healthy cells of the body. To prevent self-reactivity, thymocytes are screened in the thymus by negative thymic selection in which potential self-reactive T cells are eliminated by apoptosis to prevent autoimmunity in a process known as central tolerance. Thymic selection eliminates the vast majority of thymocytes. The remaining thymocytes leave the thymus and migrate through the bloodstream and lymphatic system to secondary lymphoid organs or tissues, such as the lymph nodes, spleen and tonsils, where they are known as mature naïve T cells.

Any mature naïve T cells that may bind to healthy cellular epitopes and escape central tolerance are rendered nonresponsive to antigen stimulation in a state known as anergy by a process known as peripheral tolerance in which their activation and function is inhibited by regulatory T cells.

Mature naïve T cells that are not self-reactive, remain inactive unless activated by an Antigen Presenting Cell (APC). APCs include dendritic cells, macrophages, Langerhans cells and B cells. Most commonly the APCs are dendritic cells that capture foreign antigens from invading pathogens, which they process and present associated with MHC proteins on their cell surface. This mechanism ensures that, among the millions of mature naïve T cells available for activation, the T cells are activated in response to the presence of specific epitopes present on pathogens infecting the host. Dendritic cells express both class I and class II MHC proteins.

Activation involves recognition and binding by the TCR of a processed foreign epitope presented in the peptide-binding groove of a Major Histocompatibility Complex (MHC) protein on the surface of the APC. The activated T cells will become either helper T cells or cytotoxic T cells depending on the co-receptors expressed at the surface of the cell. The most important and best understood of the co-receptors on T cells are CD4 and CD8 which are single-pass transmembrane proteins with extracellular domains. The co-receptors bind to non-variable parts of the MHC protein away from the peptide-binding groove. CD4 is expressed on helper T cells and binds to class II MHC proteins. CD8 is expressed on cytotoxic T cells and binds to class I MHC proteins. CD4 thus focuses helper T cell recognition on class II MHC proteins which are expressed only on APCs (including B cells), thymus epithelial cells and some others whilst CD8 focuses cytotoxic T cell recognition on class I MHC proteins which are expressed on nucleated cells. The CD co-receptors expressed on the T cells, in combination with the MHC protein classes expressed on other cells focus helper T cells on dendritic cells, macrophages, and B cells, and cytotoxic T cells on any nucleated host cell displaying a foreign peptide on a class I MHC protein.

After the successful co-recognition of foreign epitope and MHC I self-antigen, the production of cytokines by the APC stimulates cytotoxic T cell clonal proliferation and differentiation. Activated cytotoxic T cells can differentiate into effector cytotoxic T cells that target pathogens for destruction or into memory T cells that remain long-term after an infection has been eliminated and provide a rapid response to re-infection by quickly proliferating into large numbers of effector T cells.

Class I MHC proteins are expressed on the surface of all nucleated cells in the bodies of vertebrates. They also occur on platelets but not on red blood cells. Nucleated cells of the host body continuously present peptide fragments of proteins processed from within the cytoplasm on the cell surface in association with class I MHC proteins including, in infected cells, peptide fragments of pathogens. When an activated cytotoxic T cell encounters an infected cell expressing the target epitope in association with an MHC protein the T cell kills the infected cell by inducing apoptosis.

Typically, cytotoxic T cells express TCRs that bind bacterial or viral epitopes. Natural Killer (NK) cells are similar to cytotoxic T cells but can target damaged or aberrant host cells including cancer cells.

Adoptive cell therapy involves the use of immune cells, for example T cells or NK cells, to treat cancer. The cells may be engineered to express cell surface TCRs targeted to a particular protein (for example, a protein expressed at the cell surface of a cancer cell). Alternatively, in CAR T cell therapy, a chimeric antigen receptor targeted to bind to the protein of interest is engineered for expression at the T cell surface.

The TCR expressed on the cell surface is a heterodimer consisting of 2 transmembrane protein chains linked by a disulphide bond. Each chain comprises an intracellular domain, a transmembrane region, a constant region and a variable region that includes 3 hypervariable loops, known as the complementarity determining regions (CDRs). Random combination of the V, D, and J genes in the hypervariable loops, together with further diversification mechanisms (e.g. random nucleotide addition), are estimated to yield trillions of unique TCRs. In adoptive cell therapy, genes coding for TCRs including variable regions and CDRs that bind specifically to known targets (for example, a protein expressed on the surface of a cancer cell) may be transduced into T cells or NK cells to produce immunotherapy products.

In CAR T cells the heterodimer TCR structure is most commonly replaced by a single chain variable fragment (scFv) which includes an extra-cellular ligand binding domain, a spacer domain, a transmembrane domain and one or more cytoplasmic domains. A scFv can be designed to bind any target of choice and this is usually achieved by including the DNA sequences encoding the hypervariable regions of a known antibody into a vector encoding the scFv.

First-generation CARs contain a single activation domain, which in most cases is the CD3ζ cytoplasmic domain or the γ chain of Fc receptors. Second-generation CARs commonly contain an activation domain (CD3ζ or the γ chain of Fc receptors) connected to co-stimulatory domains such as CD28 and 4-1BB. Third-generation constructs typically incorporate CD3ζ with two co-stimulatory cytoplasmic domains. The extra-cellular ligand binding domain and intracellular activation domains play key roles in ligand recognition and signalling. However, the spacer and transmembrane domains can also influence CAR functions and the design of each region of the CAR can contribute to CAR T cell function, efficacy and toxicity.

Adoptive T cell therapy involves the isolation of T cells from the blood of the subject to be treated. Typically, peripheral blood mononuclear cells (PBMCs) are collected by leukapheresis. The cells are grown in culture, enriched for T cells and activated (for example by adsorption to CD3/CD28 antibody coated beads) and engineered to express a TCR or a CAR targeted to a protein expressed on the surface of cancer cells, but not on healthy cells. The most common method for gene transduction in CAR T cells employs viral vectors carrying the CAR transgene, including γ-retrovirus and lentivirus. These vectors lead to stable and durable expression of target genes by integration into the genome of host cells. However, non-viral vectors, including self-replicating non-integrative episomal S/MAR expression vectors (see, for example WO 2019/057774) or CRISPR/Cas9-mediated site-specific gene integration may also be used. The transduced CAR T cells are then expanded in culture to produce sufficient cells for therapy and infused intravenously into the patient. Whilst the efficacy of adoptive cell therapy is usually attributed to CD8 cytotoxic CAR T cells, CD4 helper CAR T cells also cause cell death both directly and by enhancing cytotoxic T cell activity through cytokine production. Use of a mixture of CD4 and CD8 CAR T cells confers superior antitumor reactivity in vivo, indicating their synergistic antitumor effect (Zhang et al. (2020) Exp. Hematol. Oncol. 9:34).

These treatments are autologous. In autologous CAR T cell therapy, the patient is treated with their own T cells that have been engineered. The use of allogeneic CAR T cell therapies, in which cancer patients are treated with fully functional healthy T cells obtained from healthy donors, would allow the engineering of “off-the-shelf” CAR T cell products with increased accessibility for patients, reduced manufacturing costs and reduced treatment costs. However, allogeneic T cells may be rejected by the host, impeding anti-tumour activity and causing graft-vs-host disease and no such products have yet been approved for clinical use.

Autologous CAR T cell therapies have proven successful in the treatment of haematological cancers including some leukaemias, lymphomas and myelomas and some CAR T cell therapies have been approved by the FDA for use in the United States including Breyanzi for adults with some types of reoccurring B cell lymphoma, Tecartus for adults with reoccurring mantle cell lymphoma, Kymriah for people under 25 years of age with leukaemia and adults with reoccurring large B cell lymphoma and Yescarta for adults with certain forms of reoccurring B cell lymphoma. One of the factors underlying this success is the selection of suitable CAR T target epitopes. A commonly targeted protein is CD19. CD19 expression on normal cells is limited to those of B cell lineage and CD19 targeted CAR T cell treatment has been demonstrated to be an effective and safe treatment target for B cell acute lymphocytic leukaemia, chronic lymphocytic leukaemia and B cell lymphoma.

However, the success rate for treating haematological cancer diseases varies and there remain a number of problems with CAR T cell therapies. Limitations of CAR T cell therapies include antigen escape, poor T cell expansion, poor T cell persistence, T cell exhaustion, and the effect of the immunosuppressive tumour microenvironment. Antigen escape involves the development of tumour resistance to therapy in a significant proportion of patients due to partial or complete loss of target antigen expression by the malignant cells of the cancer. In addition, the tumour microenvironment includes many immunosuppressive cells including myeloid-derived suppressor cells, tumour-associated macrophages, and regulatory T cells. In addition, immune checkpoint pathways such as PD-1 or CTLA-4 can decrease antitumor immunity. The resulting immunosuppressive environment may lead to CAR T cell exhaustion, poor T cell expansion and shortened T cell persistence (Zhang et al. (2020) Exp. Hematol. Oncol, 9:34).

Dual-antigen or multiantigen targeting by CARs may result in synergistic responses in cancer treatment and prevention of antigen escape. Recently, bispecific CAR T cells have been described. One bispecific CAR T cell format involves the simple mixing of two CAR T cell lines, each expressing a different scFv on its surface. A disadvantage of this approach is that the possible preferential growth of one cell line over the other, may result effectively in a single CAR T cell treatment. Another bispecific CAR T cell format involves the simultaneous transduction into T cells of two different scFv-based CAR plasmids. However, to date this approach has not proven effective. A third approach involves transducing a T cell with a single CAR plasmid that expresses two different scFvs. Therapeutics based on CAR T cells including bispecific scFv-based CARs, for example CD19/CD22 CAR T cells, have shown promising results (Dai et al. (2020) J. Hematol. Oncol. 13:30).

The most common side effect of CAR T cell therapy is Cytokine Release Syndrome (CRS) which is reported to occur in 37-93% of patients treated for lymphoma and 77-93% of patients treated for leukaemia. CRS is a potentially life-threatening condition that results from the pathologic over-activation of T cells, leading to hypersecretion of cytokines by T cells and other immune cells including IFN-γ, IL-6, IL-8, IL-10, monocyte chemoattractant protein-1 (MCP-1) and macrophage inflammatory protein-1b. The onset of CRS may range from a few hours to more than a week after CAR T cell infusion and typically resolves within 7-8 days but may persist beyond 30 days. Approximately 50% of patients enrolled in early CAR T cell trials required intensive care management for CRS (Santamasso et al. (2019) ASCO Educational Book 39:433-444)

The greatest limitation to current CAR T cell treatments is that they are not suitable for the treatment of solid tumours. The main reason for this is that suitable target epitopes that are unique to solid cancer cells and not expressed on healthy cells have not been found.

There is therefore a great need for CAR T cell therapies suitable for the treatment of solid tumours and their metastases. The present invention targets novel epitopes related to chromatin rather than cell surface proteins leading to improved adoptive cell therapies that are less susceptible to T cell exhaustion, avoid antigen escape and are applicable to solid tumours, including during metastasis.

### SUMMARY OF THE INVENTION

According to a first aspect of the invention, there is provided an immune cell comprising a chimeric antigen receptor (CAR) or a T cell receptor (TCR) wherein said CAR or TCR comprises an antigen binding domain capable of specific binding to one or more epitopes associated with neutrophil extracellular traps (NETs).

In another aspect, there is provided a method of producing the immune cell as described herein, said method comprising introducing into a cell a recombinant adeno-associated virus (rAAV) expression vector or a scaffold matrix attachment region (S/MAR) expression vector comprising a nucleic acid sequence comprising a promoter operably linked to a sequence encoding a CAR or TCR comprising an antigen binding domain capable of specific binding to one or more epitopes associated with NETs.

According to a further aspect, there is provided the immune cell as described herein for use in the treatment of a NETosis related disease.

In a yet further aspect, there is provided a method of treating a NETosis related condition in a subject in need thereof, said method comprising administering to the subject an immune cell comprising a CAR or TCR comprising an antigen binding domain capable of specific binding to one or more epitopes associated with NETs as described herein.

According to a further aspect, there is provided a method of treating a NETosis related condition in a subject in need thereof, said method comprising administering to the subject a deoxyribonuclease enzyme wherein said deoxyribonuclease enzyme is genetically engineered to contain a targeting domain capable of specific binding to one or more epitopes associated with NETs.

### BRIEF DESCRIPTION OF THE FIGURES

FIG. 1: Schematic design of nucleosome targeted CARs. VL and VH=variable domains of the anti-H3.1 and anti-H3cit antibodies, GS=Gly-Ser linker, TM=transmembrane domain, 4-1BB=cytoplasmic activation domain from CD137, CD3ζ=the cytoplasmic activation domain of CD3ζ.

FIG. 2: Structure of the resulting CAR T-cell vector plasmids. The arrangement of genes in expression cassette is in the order of: interleukin 2 signal peptide at the N terminus; IgG4 hinge CH2-CH3 spacer domain; GGGS (G4S) linker; a CD28 transmembrane and intracellular region; intracellular domains of the 4-1BB and CD3ζ.

FIG. 3: Manufacturing yield of CAR T lentiviral vectors from results of Lenti-X p24 ELISA assay. Transduction of HEK293T cells with 5 ml of concentrated CAR T lentiviruses. Infectious units (IFU) above of 10⁷/ml indicate presence of over 10¹⁰ lentiviral particles per ml. Thus lentiviral vectors coding the anti-H3.1 LgH, anti-H3.1 HgL, anti-H3cit LgH and anti-H3cit HgL CAR transgenes can be successfully manufactured.

FIG. 4: Analysis of CAR expression in T cells transduced by anti-H3.1 LgH, anti-H3.1 HgL, anti-H3cit LgH and anti-H3cit HgL CARs. T cells were labelled with anti-IgG4 antibodies. Following transduction, all CARs were efficiently expressed by transduced T cells.

FIG. 5: Cytotoxicity of a nucleosome targeted anti-H3.1 LgH, anti-H3.1 HgL, anti-H3cit LgH and anti-H3cit HgL CAR T cells. Effect of nucleosome concentrations on tumor cell lysis by CAR-T cells and Mock scFv control. U-87 MG cells were incubated in the presence of CAR-T cells plus different nucleosome concentrations or without nucleosomes prior to analysis of cell lysis (effector: tumor cell ratio 4:1).

FIG. 6: Effect of nucleosomes released from NETs in presence of DNaseI on tumor cell lysis by anti-H3.1 LgH, anti-H3.1 HgL, anti-H3cit LgH and anti-H3cit HgL CAR-T cells. Firefly luciferase expressing U-87 MG ffluc cells were incubated in the presence of CAR-T cells plus neutrophil extracellular traps (NETs; isolated from PMA induced HL-60 cells) in presence or absence of DNaseI (10 mg/ml) for 24 hours prior to analysis of luciferase activity (effector:tumor cell ratio 4:1; n=4). All data represent mean±SD.

FIG. 7: Effect of nucleosome concentrations on cytokine release by anti-H3.1 LgH, anti-H3.1 HgL, anti-H3cit LgH and anti-H3cit HgL CAR-T cells. U-87 MG cells were incubated in the presence of CAR-T cells plus different concentrations nucleosome prior to analysis of IL-2 and IFN-γ (IFNg) cytokine release (effector:tumor cell ratio 2:1). All data represent mean±SD.

FIG. 8: Typical in vivo imaging, tumor growth and survival readouts in MC38 hCEA/Ffluc-C57BL/6N model.

### DETAILED DESCRIPTION

Adoptive cell therapies typically involve T cells, NK cells or macrophage cells that express CARs or TCRs at the cell surface that are engineered to bind specifically to a target protein expressed on the surface of a cancer cell. Whilst target cancer cell surface proteins have been identified for some haematologic cancers, most cancers are not currently treated by adoptive cell therapy as suitable target proteins present on the surface of cancer cells but absent from the surface of normal cells have not been identified.

Neutrophil extracellular traps (NETs) are three-dimensional web-like structures composed of extracellular strands of decondensed chromatin expelled from activated neutrophils. NETs are a component of the innate immunity that trap and destroy pathogens locally at the site of an infection. However, excessive NETs formation may be damaging to the host tissues and is associated with a wide variety of disease states including inflammatory disease, respiratory disease (e.g. acute respiratory distress syndrome or pneumonia), cardiovascular disease, stroke and renal dysfunction as well as vascular conditions, coagulopathies and thrombosis. Thrombosis is a common complication in cancer patients, especially in metastatic cancer, and can lead to death (Demers and Wagner. Semin (2014) Thromb. Hemost., 40: 277-283; Huang et al. (2015) Hepatology, 62: 600-614; and Cedervall et al. (2018) Thromb. Res., 164(Suppl 1): S148-S152).

By contrast to the damage that can be caused to host tissues by excessive NETs production, the presence of NETs is reported to have a protective effect in cancer to shield cancer cells from attack by T cells or NK cells. Cancer cells grow in an inflammatory tumour environment that includes neutrophils and NETs. Cancer cells in the tumour environment may become wrapped in a coat of NETs. This coat of NETs shields the cancer cells from the cytotoxicity mediated by T cells and NK cells by obstructing contact between white blood cells and the cancer cells, preventing the engagement of and recognition by surface receptors such as the TCR. DNase digestion of the NETs coat has been shown to reduce or eliminate the protective effect of NETs rendering the cancer cells more susceptible to attack by T cells and NK cells (Teijeira et al. (2020) Immunity, 52). In cancer conditions both protective effects on cancer cells and tissue damaging effects are likely to occur concurrently.

It has further been reported that cancer cells may break away from a tumour in a protected NETs coat and circulate in the extracellular fluid compartment of the body facilitating the spread of tumour cells and the establishment of secondary metastases at sites that may be distant from the primary tumour. Moreover, metastatic cancer cells spread and seeded in this way may remain protected by NETs whilst they multiply and grow as secondary tumours. NETs are involved in tumour growth, invasion, and metastasis (Albrengues et al. (2018) Science, 361: eaao4227, Cools-Lartigue et al. (2013) J Clin Invest, 123: 3446-3458, and Yang et al. (2020) Nature, 583: 133-138). High levels of NETs deposition within the tumour tissue predicts poor survival and may facilitate the development of drug resistance and related toxicities (Jin et al. (2019) Ann Surg Oncol, 26: 635-643, and Mittra et al. (2017) Ann Oncol, 28: 2119-2127).

The present invention relates to a method for the treatment of a subject in need of treatment for a NETosis related disease (in particular, cancer), by an adoptive cell therapy involving an immune cell engineered to express a CAR or TCR targeted to bind to an extracellular trap, especially to a neutrophil extracellular trap (NET). This has particular advantages for the treatment of cancer over existing treatments which include:
(i) a wide range of haematological and solid cancers are amenable to the treatment because NETs are associated with most or all cancer diseases;
(ii) the treatment may lead to reduction in metastasis through direct or indirect effects on NETs associated with circulating tumour cells, such as by targeting circulating metastatic tumour cells, or through inhibition of the release of tumour cells into the circulation;
(iii) there is a reduced likelihood that cancer cells will develop treatment resistance through antigen escape mechanisms because NETs are associated with a shielding of cancer cells, rather than being expressed by cancer cells; and
(iv) treatment targeting NETs may lead to reduced thrombosis, reduced cytokine release, reduced inflammation and reduced T cell exhaustion (Sabbione et al. (2017) J Innate Immun, 9: 387-402).

Therefore, according to a first aspect of the invention there is provided an immune cell (i.e. an engineered immune cell) comprising a chimeric antigen receptor (CAR) or a T cell receptor (TCR) wherein said CAR or TCR comprises an antigen binding domain capable of specific binding to one or more epitopes associated with neutrophil extracellular traps (NETs).

Immune cells are cells involved in the immune system. In one embodiment, the immune cell is a T cell, a natural killer cell or a macrophage. In one embodiment, the immune cell is a T cell. In another embodiment, the immune cell is a natural killer (NK) cell. In another embodiment the immune cell is a macrophage. T cells that express a CAR are often referred to as CAR T cells. Similarly, NK cells that express a CAR may be referred to as CAR NK cells and macrophages that express a CAR may be referred to as CAR macrophage cells.

As extracellular traps (ETs) and NETs are comprised of chromatin, the CAR or TCR may be engineered to bind specifically to an epitope present on any chromatin protein, such as a structural chromatin protein, a high mobility group box protein, a histone modifying enzyme, a transcription factor or a histone protein (such as Histone H1, Histone H2A, Histone H2B, Histone H3 or Histone H4), deoxyribonucleic acid (DNA), or a conformational epitope present in a nucleosome (for example due to the interaction of one or more histones and/or DNA in a nucleosome). In addition, NETs chromatin may be decorated with enzymes particularly myeloperoxidase (MPO) or neutrophil elastase (NE) and such proteins adducted to NETs may also be targeted for binding by the CAR or TCR.

Thus, in one embodiment the CAR or TCR comprises an antigen binding domain capable of specific binding to one or more epitopes associated with NETs. In a further embodiment, the antigen binding domain specifically binds to one or more epitopes associated with chromatin.

The epitope targeted for binding by the CAR or TCR, such as by the antigen binding domain of the CAR or TCR, may be any epitope present in chromatin, ETs or NETs. In one embodiment the CAR or TCR, such as by the antigen binding domain of the CAR or TCR, is directed to bind a structural chromatin protein such as a Histone H1, Histone H2A, Histone H2B, Histone H3, Histone H4. In one embodiment the epitope targeted for binding by the CAR or TCR, such as by the antigen binding domain of the CAR or TCR, may be DNA. In one embodiment the epitope targeted for binding by the CAR or TCR, such as by the antigen binding domain of the CAR or TCR, may be any epitope present in a nucleosome including a conformational epitope present, for example due to the interaction of one or more histones and/or DNA. NETs are decorated with granular or cytosolic or enzyme proteins. Therefore, in one embodiment the CAR or TCR, such as the antigen binding domain of the CAR or TCR, is directed to bind a protein associated with an ET or NET including, for example MPO or NE. C-Reactive Protein (CRP) is an acute phase protein known to be physically associated with NETs (Xu et al. (2015) BMC Immunol. 16:10). Therefore, in one embodiment the CAR or TCR, such as the antigen binding domain of the CAR or TCR, is directed to bind to CRP.

As nucleosomes consist of a complex of at least 8 histone proteins (2 each of histones H2A, H2B, H3 or H4) bound to a length of DNA, a nucleosome within a NET may be bound by a CAR or TCR directed to bind to an epitope present on any nucleosome component or to a conformational epitope which may be present in a nucleosome through the interactions of nucleosome components, but absent from isolated histones or DNA.

Nucleosomes may include a wide variety of epigenetic features including different histone isoforms (or variants), different histone modifications, different nucleotide modifications (for example 5-methylcytosine or 5-hydroxymethylcytosine).

The structure of a nucleosome may vary by the inclusion of alternative histone isoforms or variants which are different gene or splice products and have different amino acid sequences. Many histone isoforms are known in the art. Histone isoforms can be classed into a number of families which are subdivided into individual types. The nucleotide sequences of a large number of histone isoforms are known and publicly available for example in the National Human Genome Research Institute NHGRI Histone Database (Mariño-Ramírez et al. The Histone Database: an integrated resource for histones and histone fold-containing proteins. Database Vol. 2011 and http://genome.nhgri.nih.gov/histones/complete.shtml), the GenBank (NIH genetic sequence) Database, the EMBL Nucleotide Sequence Database and the DNA Data Bank of Japan (DDBJ). For example, variants of histone H2 include H2A1, H2A2, mH2A1, mH2A2, H2AX and H2AZ. In another example, histone isoforms of H3 include H3.1, H3.2, H3.3 and H3t. Therefore, in one embodiment the CAR or TCR, such as the antigen binding domain of the CAR or TCR, is directed to bind to a histone isoform.

The structure of a nucleosome may also vary by post translational modification (PTM) of histone proteins. Known post translational modifications include acetylation, methylation, which may be mono-, di- or tri-methylation, phosphorylation, ribosylation, citrullination, ubiquitination, hydroxylation, glycosylation, nitrosylation, glutamination and/or isomerisation (Ausio et al. (2001) Biochem Cell Biol. 79: 693-708). PTM of histone proteins typically occurs on the tails of the core histones and common modifications include acetylation, methylation or ubiquitination of lysine residues as well as methylation or citrullination or arginine residues and many others. Many histone modifications are known in the art and the number is increasing as new modifications are identified (Zhao and Garcia (2015) Cold Spring Harb Perspect Biol, 7: a025064). Therefore, in one embodiment the CAR or TCR is directed to bind to a modified histone.

In some preferred embodiments the antigen binding domain of the CAR comprises an scFv. The employed scFv might be derived from an antibody specific to histone H3.1 or citrullinated histone H3 (H3cit). In some preferred embodiments, the scFv comprises heavy chain and light chain variable region sequences derived from an anti-H3.1 or anti-citrullinated H3 antibody and optionally connected with a Ser-Gly linker. Therefore, in one embodiment, the scFv comprises two variable (V) fragments linked via a Ser-Gly linker having the sequence GGGGSGGGGSGGGGS (SEQ ID NO:6).

As exemplified, the employed scFv fragment can be connected to the other domains of the CAR of the CAR-T cell via standard recombinant techniques well known in the art. In some embodiments, the scFv is conjugated to the transmembrane domain of the CAR via a spacer or hinge domain to provide flexibility and to facilitate interaction of the scFv with its substrate. In some embodiments, the spacer domain comprises CH2-CH3 IgG4 as exemplified herein. Other suitable spacer or hinge domains for connecting the scFv to the transmembrane domain are also exemplified below.

-CH2-CH3 IgG4 spacer domain:
APEFEGGPSVFLFPPKPKDTLMISRTPEVTCVVVDVSQEDPEVQF
NWYVDGVEVHNAKTKPREEQFQSTYRVVSVLTVLHQDWLNGKEYK
CKVSNKGLPSSIEKTISKAKGQPREPQVYTLPPSQEEMTKNQVSL
TCLVKGFYPSDIAVEWESNGQPENNYKTTPPVLDSDGSFFLYSRL
TVDKSRWQEGNVFSCSVMHEALHNHYTQKSLSLSLGK
(SEQ ID NO: 7)
IgG4 hinge:
ESKYGPPCPSCP
(SEQ ID NO: 8)
CD8a hinge domain:
TTTPAPRPPTPAPTIASQPLSLRPEACRPAAGGAVHTRGLDFAC
(SEQ ID NO: 9):
IgG4 CH2 domain:
APEFEGGPSVFLFPPKPKDTLMISRTPEVTCVVVDVSQEDPEVQF
NWYVDGVEVHNAKTKPREEQFQSTYRVVSVLTVLHQDWLNGKEYK
CKVSNKGLPSSIEKTISKAK
(SEQ ID NO: 10)
IgG4 CH3 domain:
GQPREPQVYTLPPSQEEMTKNQVSLTCLVKGFYPSDIAVEWESNG
QPENNYKTTPPVLDSDGSFFLYSRLTVDKSRWQEGNVFSCSVMHE
ALHNHYTQKSLSLSLGK
(SEQ ID NO: 11)

In some embodiments, there can be a second linker between the hinge or spacer and the transmembrane to provide additional flexibility. An example of such a second linker, GSTSGSGKPGSGEGSTKG (SEQ ID NO:12), may be present in the CAR.

Transmembrane and intracellular domain sequences that are suitable for constructing the CAR molecules of the invention are well known in the art. These include the CD28 transmembrane and intracellular domain, OX-40 intracellular domain, and the CD3zeta (CD3ζ) intracellular domain, as exemplified herein. Construction of the CAR molecules of the invention with the various component motifs or domains can be readily performed in accordance with standard recombinant techniques well known in the art or specific guidance described herein.

CD28 transmembrane and intracellular domain:
(SEQ ID NO: 13)
FWVLVVVGGVLACYSLLVTVAFIIFWVRSKRSRLLHSDYMNMTPR
RPGPTRKHYQPYAPPRDFAAYRS.
CD28 intracellular domain:
(SEQ ID NO: 14)
RSKRSRLLHSDYMNMTPRRPGPTRKHYQPYAPPRDFAAYRS.
OX-40 intracellular domain:
(SEQ ID NO: 15)
RDQRLPPDAHKPPGGGSFRTPIQEEQADAHSTLAKI.
4-1BB intracellular domain:
(SEQ ID NO: 16)
KRGRKKLLYIFKQPFMRPVQTTQEEDGCSCRFPEEEEGGCEL.
CD37 intracellular domain:
(SEQ ID NO: 17)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR.

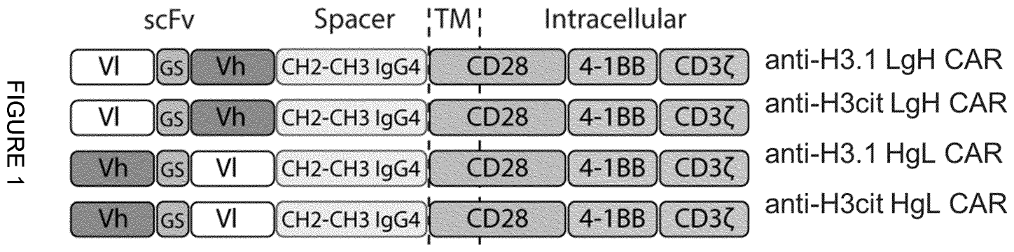

In one embodiment, the CAR comprises the components as depicted in FIG. 1.

A major pathway for the formation of NETs by NETosis involves PAD4 (peptidyl arginine deiminase 4) which is an enzyme that converts arginine to citrulline. NETs may comprise citrullinated histones and selection of citrullinated nucleosomes as a target for a CAR or a TCR may confer NETs specificity to a CAR T cell over binding to other chromatin. Therefore, in a further embodiment the CAR or TCR is directed to bind to a citrullinated histone such as H3 citrulline (H3cit) or H4 citrulline (H4cit). In a yet further embodiment, the CAR or TCR is directed to bind to histone H3 citrullinated at any of amino acids R2, R8, R17, R26 or any combination of thereof.

A group or class of related histone post translational modifications (rather than a single modification) may also be targeted. For example, pan-citrullinated, pan-acetylated or pan ubiquitinated histones may be targeted.

The structure of a nucleosome may also vary in nucleotide and modified nucleotide composition. Nucleosomes may comprise 5-methylcytosine residues or 5-hydroxymethylcytosine residues (or other nucleotides or modified nucleotides). Therefore, in a further embodiment, the CAR or TCR is directed to bind to DNA or to modified DNA. In one embodiment, the DNA modification is selected from 5-methylcytosine or 5-hydroxymethylcytosine.

The component of a nucleosome targeted for CAR or TCR binding may be a histone protein (i.e. histone H1, H2A, H2B, H3 or H4), a histone post-translational modification, a histone variant or isoform, DNA, a modified nucleotide associated with the nucleosome or a protein bound to the nucleosome as described herein.

Thus, in some embodiments the CAR or TCR, such as the antigen binding domain of the CAR or TCR, specifically binds to an epigenetic feature of a nucleosome, DNA, histone H2A, histone H2B, histone H3, histone H4 or histone H1. In a further embodiment, the epigenetic feature comprises a histone isoform or one or more post-translational histone modification.

In a yet further embodiment, the one of more post-translational histone modification comprises at least one modification selected from: N-acetylation of serine or alanine; phosphorylation of serine, threonine or tyrosine; N-acylation of lysine (e.g. crotonylation or butyrylation); N6-methylation, N6,N6-dimethylation, or N6,N6,N6-trimethylation of lysine; omega-N-methylation, symmetrical-dimethylation or asymmetrical-dimethylation of arginine; citrullination of arginine; ubiquitinylation of lysine; sumoylation of lysine; O-methylation of serine and threonine; phosphorylation of serine, threonine or tyrosine; and ADP-ribosylation of arginine, aspartic acid or glutamic acid.

Other non-chromatin proteins associated with NETs, such as MPO or NE, may also be targeted and/or specifically bound by the CAR or TCR, such as by the antigen binding domain of the CAR or TCR.

In addition, histone components of nucleosomes are subject to clipping in which the histone tail is physically and irreversibly removed by regulated proteolysis, or clipping. Furthermore, histone degradation has been shown to be involved in the formation of NETs. On histone H3, clipping is reported to occur around amino acid position 21 (Yi and Kim (2018) BMB Rep 51: 211-218). We conclude that CARs or TCRs directed to bind to histone epitopes that may be removed by clipping, may fail to bind to nucleosomes that include clipped histones. We therefore selected CARs directed to bind to core histone and nucleosome epitopes.

In a particular embodiment the CAR or TCR of the invention, such as the antigen binding domain of the CAR or TCR, is directed to bind to a core histone/nucleosome epitope not subject to clipping. The amino acid sequence of histone H3.1, H3.2 and H3t at positions 27-36 is KSAPATGGVK (SEQ ID NO: 1). The amino acid sequence at positions 29-35 (APATGGV [SEQ ID NO: 2]) does not include any commonly post-translationally modified amino acids (for example lysine, serine or arginine). Therefore, antibodies directed to bind to this epitope (i.e. amino acid positions 29-35) are unaffected, or minimally affected, by the post-translational modification status of the nucleosome, and will bind to all or most nucleosomes containing histone H3.1, regardless of PTM structure. Therefore, we designed plasmids for CARs that include antigen binding domains directed to bind to amino acid position 30-33 of histone H3.1 so that both intact and clipped nucleosomes are bound by the CARs and TCRs regardless of their PTM status to maximise the binding of nucleosomes and NETs. The amino acid sequence of histone H3.1 is known in the art and is described at UniProt Accession No. P68431.

Therefore, in one embodiment of the invention, the CAR or TCR, such as the antigen binding domain of the CAR or TCR, is directed to bind to a core histone epitope of histone H3.1 at an amino acid epitope located higher than amino acid position 21. In a particular embodiment, the CAR or TCR, such as the antigen binding domain of the CAR or TCR, is directed to bind to an epitope located at or near to amino acid position 29-35, in particular at or near to amino acid position 30-33.

We also designed plasmids for CARs directed to bind to a conformational nucleosome epitope present in intact nucleosomes but not present on isolated (free) histone or DNA nucleosome components. This type of epitope may be referred to as a “conformational nucleosome epitope” herein because it requires the native three-dimensional configuration of the target nucleosome to be intact. We reasoned that the use of this epitope would similarly maximise binding of intact nucleosomes in NETs by CAR T cells. Therefore, in one embodiment the CAR or TCR, such as the antigen binding domain of the CAR or TCR is directed to bind to a conformational nucleosome epitope.

We also designed plasmids for CARs directed to bind to a nucleosome containing histone H3 citrullinated at the arginine at amino acid position 8 (H3R8Cit) as we reasoned the use of this epitope would maximise the specificity of CAR T cells for chromatin in NETs over other forms of cell free chromatin. Therefore, in one embodiment the CAR or TCR, such as the antigen binding domain of the CAR or TCR, is directed to bind to H3R8Cit.

CAR T cell therapy is effective and widely used. Methods for generation of CAR T cell therapies are well known in the art (see for example Fesnak and O'Dohrty (2017) European Oncology & Haematology, 13: 28-34). Without limitation, in a typical CAR T cell of the invention, CDRs that bind specifically to a nucleosome or NETs epitope target described herein are inserted into a plasmid as a cassette encoding a scFv or TCR in combination with an operably linked promoter and transduced into activated T cells. Single or multiple promoters may be used. Example promoters include CMV, EF-1, hPGK and RPBSA. Methods for the preparation of scFv cassettes, plasmids and vectors for the production of CAR T cells are well known in the art (see for example: Labanieh et al. (2018) Nat Biomed Eng 2: 377-391, Larson and Maus (2021) Nat Rev Cancer 21: 145-161, Carl et al. (2018) Science 359, 1361-1365, and Rad et al. (2020) PLoS ONE 15(7): e0232915). The vector selected for transduction is typically a viral vector, for example an adeno-associated virus (AAV) vector or a retroviral or lentiviral vector due to the high efficiency of gene delivery and the persistence of integrating vectors in the modified T cells.

Thus, in some embodiments a sequence encoding the CAR or TCR described herein is introduced into the immune cell using a recombinant adeno-associated virus (rAAV) expression vector. Therefore, in one aspect, there is provided an expression vector comprising a sequence encoding the CAR or TCR described herein. In one embodiment, the vector is an rAAV comprising a capsid protein and a nucleic acid sequence comprising a promoter operably linked to a sequence encoding the CAR or TCR directed to bind to NETs as described herein.

The vector may comprise sense or anti-sense single stranded DNA or double stranded (self-complementary) DNA. Optionally other genes may also be incorporated into the vector, for example a gene encoding for antibiotic resistance.

Many examples of promoters, capsid proteins, vectors and enhancers suitable for use in the present invention are known in the art (see for example WO2019143272 and WO2019057774, the contents of which are hereby incorporated). Non-limiting examples of promoters which can be used in the vectors of the invention include, for example, an albumin promoter, an al-anti-trypsin (AAT) promoter, a thyroid hormone-binding globulin promoter, an alpha fetoprotein promoter, an alcohol dehydrogenase promoter, a factor VIII (FVIII) promoter, a HBV basic core promoter (BCP), a HBV PreS2 promoter, a phosphoenol pyruvate carboxykinase (PEPCK) promoter, a thyroxin-binding globulin (TBG) promoter, an Hepatic Control Region (HCR)-ApoCII hybrid promoter, an HCR-hAAT hybrid promoter, an apolipoprotein E (ApoE) promoter, a low density lipoprotein promoter, a pyruvate kinase promoter, a phosphenol pyruvate carboxykinase promoter, a phenylalanine hydroxylase promoter, a lecithin-cholesterol acyl transferase (LCAT) promoter, an apolipoprotein H (ApoH) promoter, an apolipoprotein A-II promoter (APOA2), a transferrin promoter, a transthyretin promoter, an a-fibrinogen promoter, a b-fibrinogen promoter, an alpha I-antichymotrypsin promoter, an a2-HS glycoprotein promoter, an haptoglobin promoter, a ceruloplasmin promoter, a plasminogen promoter, a promoter of a complement protein, al-acid glycoprotein promoter, a LSP1 promoter, a serpin peptidase inhibitor promoter, a clade A member 1 (SERPINA1) (hAAT) promoter, a Cytochrome P450 family 3 subfamily A polypeptide 4 (CYP3A4) promoter, a microRNA 122 (miR-I22) promoter, a liver-specific IGF-II promoter PI, a transthyretin (MTTR) promoter, and an a-fetoprotein (AFP) promoter.

Non-limiting examples of capsid proteins, include wherein the capsid protein comprises one or more mutations selected from the group consisting of S279A, S671A, K137R, T252A, and any combinations thereof. In some embodiments, the one or more mutations in the capsid protein are derived from an AAV such as Anc80 or Anc80L65. In other embodiments, the capsid protein is a mutant AAV8 capsid protein such as AAV3G1, AAVT20 or AAVTR1, or another mutant capsid protein disclosed in WO2017/180854 (e.g. comprising VP3 mutations in amino acids 263-267, and/or amino acids 455-459), the contents of which are hereby incorporated.

Non-limiting examples of AAVs which can be used in the vectors of the invention include, e.g. AAV serotype 1 (AAV1), AAV2, AAV3 (including AAV3A and AAV3B), AAV4, AAV5, AAV6, AAV7, AAV8, AAV9, AAV10, AAV11, AAV12, AAVrhIO, AAVLK03, AAVhu37, AAVrh64RI, and Anc 80. In one embodiment, the AAV is from serotype 8 (AAV8) or Anc 80.

In one embodiment, the nucleic acid of the AAV vector further comprises one or more enhancers located upstream or downstream of the promoter. In one embodiment, the enhancer may be located immediately upstream with the promoter, e.g. where the 3′ end of an enhancer sequence fused directly to the 5′ end of the promoter sequence. Non-limiting examples of useful enhancers include, e.g. an apolipoprotein E (ApoE) enhancer (e.g. an ApoE hepatic control region-I (HCR-I) enhancer or an ApoE HCR-2 enhancer), an alpha fetoprotein enhancer, a TTR enhancer, an LSP enhancer, an al-microglobulin/bikunin enhancer, an albumin gene enhancer (Ealb), and any combination thereof. In some embodiments, the enhancer is an ApoE enhancer. In one embodiment, the enhancer is an ApoE enhancer located upstream of the promoter. In a further specific embodiment, the enhancer is an ApoE enhancer fused to the 5′ end of the promoter. In a yet further embodiment, the ApoE enhancer is a hepatic control region (HCR) enhancer.

In one embodiment, the nucleic acid of the AAV vector further comprises a polyadenylation signal operably linked to the nucleotide sequence encoding the CAR. In one embodiment, the nucleic acid further comprises a Kozak sequence. In one specific embodiment, the Kozak sequence comprises the sequence of 5′-GCCGCCACC-3′ or similar.

In one embodiment, the nucleic acid further comprises a post-transcriptional regulatory element. In one embodiment, the post-transcriptional regulatory element is a woodchuck hepatitis post-transcriptional regulatory element (WPRE).

In a typical CAR- or TCR-expressing cell, the nucleotide sequence encoding the CAR or TCR is integrated into the genome of a transduced T or NK cell. This method has proved successful for cancer treatment. However, randomly integrating vectors such as gamma retroviruses or transposons suffer from disadvantages including an inherent potential genotoxic risk, complicated vector-engineering to optimise gene expression and delivery as well as slow and expensive CAR T cell manufacture. In a further embodiment of the invention, the vector produced for transduction of activated T cells is a non-viral and/or non-integrating vector. An example of a non-viral, non-integrating DNA vector platform is based on scaffold/matrix attachment region (S/MAR) motifs. S/MARs are 60-5000 base pair DNA sequences that anchor chromatin to nuclear matrix proteins during interphase. S/MARs can be efficiently transfected into primary human T cells without toxicity leading to the production of 10⁹ CAR T cells in 5 days (Bozza et al. (2021) Science Advances, 17).

Therefore, in a further aspect of the invention there is provided an immune cell, such as a T cell, comprising a non-integrated DNA sequence encoding a CAR or TCR directed to bind, such as comprising an antigen binding domain capable of specific binding, to one or more epitopes associated with a nucleosome or NETs. In one embodiment said non-integrated DNA sequence is transduced by means of a non-viral vector. In one embodiment the non-viral vector is an S/MAR.

In a yet further aspect, there is provided a method of producing the immune cell as described herein, comprising introducing an expression vector encoding the CAR or TCR into the immune cell. The method may comprise introducing into the immune cell a recombinant adeno-associated virus (rAAV) expression vector comprising:
(i) a capsid protein; and
(ii) a nucleic acid sequence comprising a promoter operably linked to a sequence encoding a CAR or TCR comprising an antigen binding domain capable of specific binding to one or more epitopes associated with NETs as defined herein.

Alternatively, the method may comprise introducing into an immune cell a scaffold matrix attachment region (S/MAR) expression vector comprising a nucleic acid sequence comprising a promoter operably linked to a sequence encoding a CAR or TCR comprising an antigen binding domain capable of specific binding to one or more epitopes associated with NETs as described herein.

In some embodiments, the rAAV or S/MAR expression vector comprises a tissue specific promoter. In one embodiment, the rAAV or S/MAR expression vector includes a liver-specific promoter.

### Combination Therapies

We and others have reported that nuclease enzymes that digest DNA are effective in the treatment of cancer both in vitro and in vivo through the digestion of NETs. (Xia et al. (2020) Mol Oncol. 14: 2920-2935). Deoxyribonuclease I (DNase I) is an endonuclease that selectively cleaves the phosphodiester bond in DNA and can be used to digest the DNA scaffold of NETs. Intravenous or intraperitoneal injections of DNase I display potent antimetastatic activity and meaningful suppression of primary tumour growth in multiple animal tumour models (Alekseeva et al. (2020) Mol Ther Nucleic Acids 20: 50-61, Salganik et al. (1967) Nature 214: 100-102, Sugihara et al. (1993) Br J Cancer 67: 66-70, Tohme et al. (2016) Cancer Res March, 76: 1367-1380, and Yazdani et al. (2019) Cancer Res, 79: 5626-5639).

Similarly, a DNase 1 gene therapy involving intravenous injection of an adeno-associated virus (AAV) plasmid vector expressing a human DNase I transgene under the control of a liver-specific promoter inhibits the development of CRC liver metastasis. Digestion of NETs with DNase leads to rapid release of mononucleosomes in close proximity to cancer cells. The approach of mononucleosomes to cancer cell membranes can trigger cancer cell apoptosis. The use of DNase with checkpoint inhibitors has also been shown to reduce tumour load, tumour associated macrophages, tumour infiltrating neutrophils and to reduce or prevent CAR T cell exhaustion and systemic immunosuppression.

Thus, in one embodiment the immune cell described herein additionally comprises a deoxyribonuclease (DNase) enzyme. In a further aspect, there is provided a combination therapy comprising the immune cell described herein and a DNase enzyme. The combination therapy may involve administration of a CAR T cell therapy targeted to an epitope associated with a NET in combination with a recombinant enzyme capable of the digestion of DNA. This combination provides a double focus on NETs and cancer associated NETs through the innate immune system in combination with enzyme digestion. In some embodiments, the enzyme is a DNase enzyme selected from DNase I, DNase X, DNase γ, DNase1L1, DNase1L2, DNase 1L3, DNase II, DNase IIα, DNase IIβ, Caspase-activated DNase (CAD), Endonuclease G (ENDOG), Granzyme B (GZMB), and mutants or derivatives thereof. In some embodiments an actin resistant DNase mutant is used to overcome inhibition by actin in environments with high levels of cell death. In one embodiment the DNase is derived from a catalytic antibody having DNase activity.

In a further aspect, the DNase enzyme capable of digestion of DNA and NETs is delivered as a gene therapy for secretion into the extracellular milieu for the digestion of NETs. Therefore, the immune cell may additionally comprise a nucleotide sequence encoding a DNase enzyme. In some embodiments the nucleotide sequence encoding the enzyme comprises an additional nucleotide sequence encoding a secretory signal. The secretory signal mediates secretion of the enzyme. Thus, in one embodiment the deoxyribonuclease enzyme is secreted. Suitable secretory sequences may include, without limitation, a DNase I secretory signal sequence, an IL-2 secretory signal sequence, an albumin secretory signal sequence, a β-glucuronidase secretory signal sequence, an alkaline phosphatase secretory signal sequence or a fibronectin secretory signal sequence. Example amino acid secretory sequences include the sequence MRGMKLLGALLALAALLQGAVS [SEQ ID NO: 3], MYRMQLLSCIALSLALVTNS [SEQ ID NO: 4] and MRYTGLMGTLLTLVNLLQLAGT [SEQ ID NO: 5]. It will be understood that secretory signals such as these may be used or modified sequences with at least 80%, 85%, 90% or 95% identity with these sequences may also be used.

In one embodiment of the invention, there is provided an immune cell as described herein (i.e. wherein the CAR or TCR is directed to bind to NETs) for use in combination with a cell that secretes an enzyme capable of digesting NETs. In another embodiment, there is provided a method of treatment for a cancer comprising administering to a subject an immune cell as described herein in combination with a cell that secretes an enzyme capable of digesting NETs. Thus, in a further embodiment, there is provided an immune cell as described herein for use in combination with a DNase enzyme. In a yet further embodiment, there is provided a method of treatment for cancer comprising administering to a subject an immune cell as described herein in combination with a DNase enzyme.

Vectors including DNA encoding the DNase enzyme may be transduced into activated T cells to produce T cells expressing a DNase enzyme, for example at the cell surface. Such enzyme bearing T cells may be used in combination with (other) CAR T cells targeted to bind to NETs for use as a combined adoptive cell therapy in which some T or NK cells bind to NETs and other T or NK cells digest NETs. The DNA sequence encoding the enzyme for expression on the cell surface of a T cell or NK cell may be included in a vector for transduction of the cell, for example as part of a scFv in place of one or more, or all, CDRs. Thus, in one embodiment the deoxyribonuclease enzyme is encoded by a gene therapy vector.

In a further embodiment of the invention there is provided an immune cell as described herein wherein the CAR or TCR is directed to bind to NETs for use in combination with an immune cell, such as a T cell or NK cell, that expresses a DNase enzyme capable of digesting NETs at or near to the cell surface. Thus, in a further embodiment the deoxyribonuclease enzyme is located at or near to the cell membrane. In another embodiment there is provided a method of treatment for a cancer comprising administering to a subject an immune cell as described herein wherein the CAR or TCR is directed to bind to NETs in combination with an immune cell, such as a T cell or NK cell, that expresses a cell surface DNase enzyme capable of digesting NETs.

In some embodiments, the enzyme is a DNase enzyme selected from DNase I, DNase X, DNase γ, DNase1L1, DNase1L2, DNase 1L3, DNase II, DNase IIα, DNase IIβ, Caspase-activated DNase (CAD), Endonuclease G (ENDOG), Granzyme B (GZMB), and mutants or derivatives thereof. In some embodiments an actin resistant DNase mutant is used to overcome inhibition by actin in environments with high levels of cell death. In a further embodiment, the DNase enzyme comprises an amino acid sequence having at least 90% sequence identity to human DNase I enzyme, for example as described in UniProt ID No.: P24855 and NCBI Reference Sequence: NP_005214.2 which are herein incorporated by reference. In a yet further embodiment, the DNase enzyme comprises an amino acid sequence having at least 90% sequence identity to amino acids 21 to 305 of DNase1-like 3 (D1L3) enzyme, for example as described in UniProt ID No.: Q13609 and NCBI Reference Sequence: NP_004935.1 which are herein incorporated by reference.

Alternatively, the DNA sequence encoding the DNase enzyme together with a DNA sequence encoding a secretory sequence, may be transduced into the same immune cell comprising a CAR or TCR as described herein, such as an activated T cell comprising a scFv or TCR targeted to bind to NETs. This produces a combined CAR T cell that expresses a NETs-binding scFv or TCR at the surface of the cell and also secretes an enzyme capable of digesting the DNA component of the NETs. These cells of the invention provide a combined adoptive cell therapy that both binds to NETs and digest NETs in a single cell. Thus, in some embodiments the immune cell that expresses a DNase enzyme is the same immune cell comprising a CAR or TCR as described herein. In a further embodiment, there is provided a method of treatment for cancer comprising administering to a subject an immune cell comprising a CAR or TCR as described herein, wherein the immune cell additionally expresses a Dnase enzyme. In a yet further embodiment, there is provided an immune cell comprising a CAR or TCR as described herein and additionally expressing a Dnase enzyme, for use in a method of treating cancer.

In one embodiment, the DNase enzyme is genetically engineered to contain a targeting domain capable of specific binding to one or more epitopes associated with NETs. Therefore, if the DNase enzyme is secreted, it will contain an additional signal that targets the enzyme to NETs.

In one embodiment, the DNase and CAR or TCR amino acid sequences may be encoded within a vector for the expression of a single bi-functional CAR, TCR or other cell surface protein. Thus, in one embodiment, the DNase and CAR or TCR sequence may be encoded by the same vector. In another embodiment, the DNase and CAR or TCR sequence may be encoded by separate vectors.

In further embodiments, the method of producing the immune cell as described herein comprises introducing into a cell an rAAV or S/MAR, wherein the rAAV or S/MAR expression vector additionally comprises a nucleic acid sequence comprising a promoter operably linked to a sequence encoding an enzyme which has DNase activity. Alternatively, the sequence encoding an enzyme which has DNase activity may be introduced into the immune cell using a separate vector. Therefore, the method of producing the immune cell as described herein may comprise introducing into a cell (i) an rAAV or S/MAR expression vector comprising a nucleic acid sequence comprising a promoter operably linked to a sequence encoding a CAR or TCR comprising an antigen binding domain capable of specific binding to one or more epitopes associated with NETs; and (ii) an rAAV or S/MAR expression vector comprising a nucleic acid sequence comprising a promoter operably linked to a sequence encoding an enzyme which has DNase activity

In a further aspect of the invention there is provided a bispecific immune cell comprising a CAR or TCR as described herein targeted to bind more than one separate NETs epitopes (i.e. a “bispecific CAR/TCR”) to maximise the specificity of the immune cell for NETs over other forms of chromatin. In a preferred embodiment there is provided an immune cell comprising a bispecific CAR or TCR comprising two or more antigen binding domains capable of binding to two or more epitopes associated with NETs.

As the immune cells and methods described herein facilitate the targeting and or digestion of NETs, it will be understood that this will lead to the unmasking of cell surface proteins on the surface of a cancer cell. Such unmasked surface proteins may also be targeted by a CAR or TCR. Therefore, in a further aspect of the invention there is provided a bispecific immune cell comprising a CAR or TCR as described herein capable of binding to an epitope on NETs as well as to a protein on the surface of a cancer cell. Thus, in one embodiment there is provided an immune cell comprising a bispecific CAR or TCR comprising two or more antigen binding domains capable of binding to an epitope on NETs and an epitope on the surface of a cancer cell.

In a yet further aspect there is provided a bifunctional CAR T cell that expresses a DNase enzyme as well as a binder of a protein on the surface of a cancer cell.

### Therapeutic Uses and Methods

In one aspect of the invention, there is provided a method for the treatment of a NETosis related condition in a subject in need thereof, by an adoptive cell therapy involving an immune cell comprising a CAR or a TCR as described herein targeted to bind to an extracellular trap (ET), for example a neutrophil extracellular trap. In a further aspect, there is provided the immune cell comprising a CAR or TCR as described herein for use in the treatment of a NETosis related condition.

In some embodiments, the treatment of a NETosis related condition additionally comprises administration of a deoxyribonuclease (DNase) enzyme as described herein, such as by combined administration of an immune cell expressing a DNase enzyme or by the immune cell comprising the CAR or TCR as described herein additionally expresses a DNase enzyme.

In one embodiment, the treatment is an autologous treatment comprising the steps of:
(i) obtaining an immune cell from a subject;
(ii) engineering the immune cell obtained in step (i) to express a CAR or TCR comprising an antigen binding domain capable of specific binding to one or more epitopes associated with neutrophil extracellular traps (NETs); and
(iii) administering the engineered immune cell to the subject.

Allogeneic adoptive cell therapies involve the engineering of immune cells, such as T cells, obtained from a (healthy) donor for use as treatment (i.e. as ‘off the shelf’ therapies). Therefore, in an alternative embodiment the treatment is an allogeneic treatment comprising the steps of:
(i) obtaining an immune cell from a donor;
(ii) engineering the cell obtained in step (i) to express a CAR or TCR comprising an antigen binding domain capable of specific binding to one or more epitopes associated with neutrophil extracellular traps (NETs); and
(iii) administering the engineered immune cell to a subject in need thereof.

Administration may be by any method known in the art for cellular therapies, in particular adoptive cellular therapies. In one embodiment, administration of the immune cell is via fibroin scaffolds.

According to a further aspect, there is provided a method of treating a NETosis related condition in a subject in need thereof, said method comprising administering to the subject a deoxyribonuclease enzyme wherein said deoxyribonuclease enzyme is genetically engineered to contain a targeting domain capable of specific binding to one or more epitopes associated with NETs.

Recently, methods for the therapeutic extracorporeal removal of NETs from the circulation have been described (for example, see WO2019053243). This therapy is synergistic with all of the immune cells comprising a CAR or TCR, including engineered therapeutic cells and adoptive cell therapies, described herein. Therefore, in further aspects of the invention there is provided a combined adoptive cell therapy targeted at NETs in combination with an extracorporeal NETs removal therapy.

Adoptive cell therapies of the invention may be used for the treatment of cancer as described herein. However, excessive production of NETs is pathological and is additionally involved in a long and continually growing list of disease processes including, without limitation, sepsis, COVID-19, acute respiratory distress syndrome, severe acute respiratory syndrome, all autoimmune conditions, all inflammatory conditions, Alzheimer's disease, atherosclerosis, bacterial infection, cystic fibrosis, pancreatitis, viral infection, diabetes, thrombosis, disseminated intravascular coagulation, pneumonia, respiratory infections, coronary thrombi, stroke, deep vein thrombosis, gout and many more (Sollberger et al. (2018) Developmental Cell 44(5):542-553, Thalin et al. (2019) Arterioscler. Thromb. Vasc. Biol. 39:1724-1738, Neubert et al. (2019) Frontiers in Immunology 10:12). Therefore, adoptive cell therapies of the invention may be used for the treatment of any NETosis related condition. In preferred embodiments, adoptive cell therapies of the invention are used for the treatment of life-threatening conditions, including cancer, sepsis, COVID-19 and other acutely life-threatening NETosis related conditions. Thus, in one embodiment the NETosis related condition is sepsis, an inflammatory condition or cancer. In a particular embodiment, the cancer is a metastatic cancer.

It will be understood that the embodiments described herein may be applied to all aspects of the invention, i.e. the embodiment described for the uses may equally apply to the claimed methods and so forth.

The invention will now be illustrated with reference to the following non-limiting examples.

### EXAMPLES

### Example 1: Design of the CAR Molecules Having Antigen Binding Domain Capable of Specific Binding to One or More Epitopes Associated With Neutrophil Extracellular Traps (NETs)

Nucleosomes, the basic units of chromatin and neutrophil extracellular traps, consist of DNA wrapped around a core of eight histone proteins composed of two dimers of histones H2A and H2B and a tetramer of histones H3 and H4. PAD4-mediated hypercitrullination of histone H3 is critical for the neutrophil extracellular trap (NET) formation. During NETosis, histone hypercitrullination promotes chromatin unravelling and chromatin complex extrusion from the cell to form NETs. Nucleosomes forming NETs contain histone H3.1 in their octamer and citrullinated histone H3.1 is a specific epigenetic feature of NETs. To construct CAR molecules having an antigen binding domain capable of specific binding to one or more epitopes associated with neutrophil extracellular traps (NETs) we generated four scFv fragments derived from two monoclonal antibodies raised against histone H3.1 and epigenetically modified citrullinated histone H3. The scFv targeting histone H3.1 (scFv-H3.1) contains a V_{H} fragment and a V_{K} fragment linked via a Ser-Gly linker (SEQ ID NO:6). The scFv targeting citrullinated histone (scFv-H3cit) contains a V_{H} fragment and a V_{L} fragment linked via a Ser-Gly linker (SEQ ID NO:6). Four second generation CAR molecules were engineered by fusing scFv-H3.1 and scFv-H3cit with V_{H} and V_{L} fragments arranged in different orders to the IgG4 hinge region, CD28 transmembrane and intracellular region, and CD3ζ with intracellular costimulatory domain of 4-1BB. These were termed anti-H3.1 LgH, anti-H3.1 HgL, anti-H3cit LgH and anti-H3cit HgL, depending on the target (H3.1 or H3cit) and the order of VH/VL fragments). Schematic designs of the resulting CAR molecules are presented in FIG. 1.

### Example 2: Manufacture of Lentiviral Vectors Encoding Anti-Nucleosome CAR Molecules

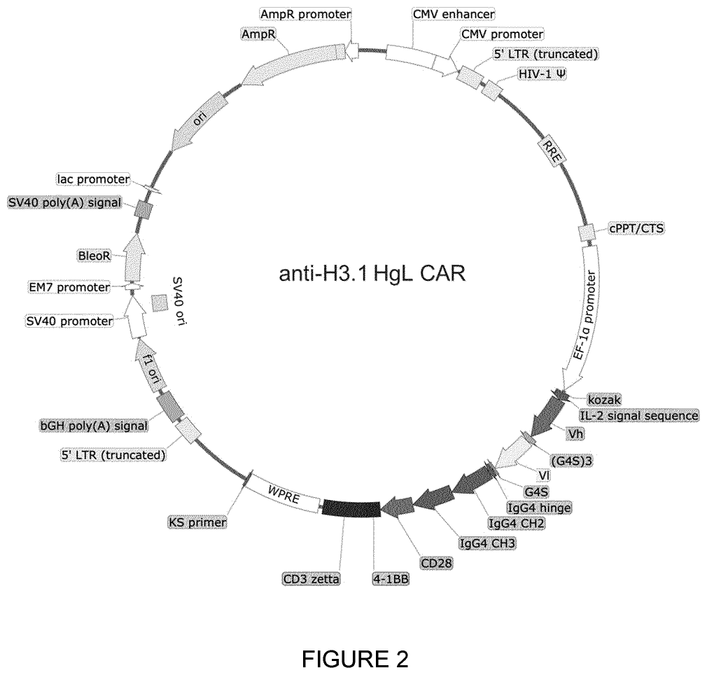

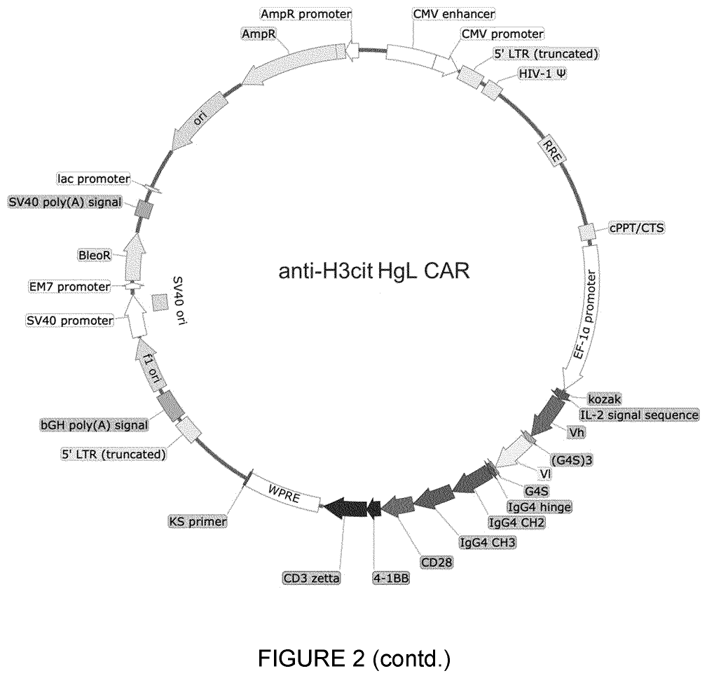

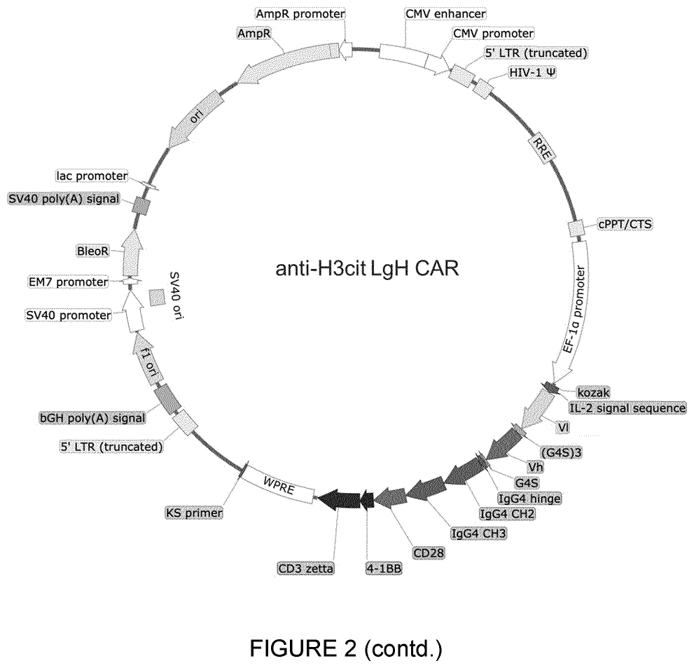

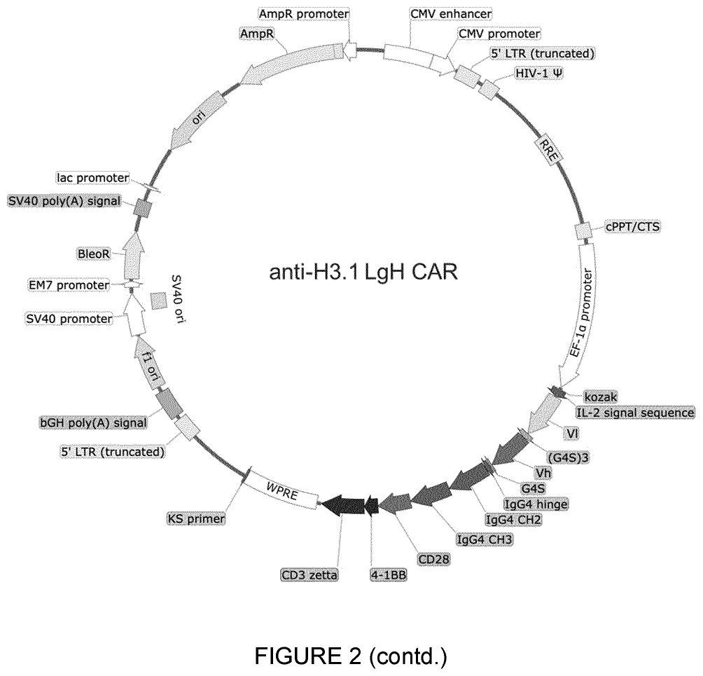

The DNA fragments coding for the anti-H3.1 LgH, anti-H3.1 HgL, anti-H3cit LgH and anti-H3cit HgL CAR T-cell receptors were synthesized and cloned into the pLV2 lentiviral vector (Clontech) under the control of the EF1a promoter. The structure of the resulting CAR T-cell vector plasmids are presented in FIG. 2.

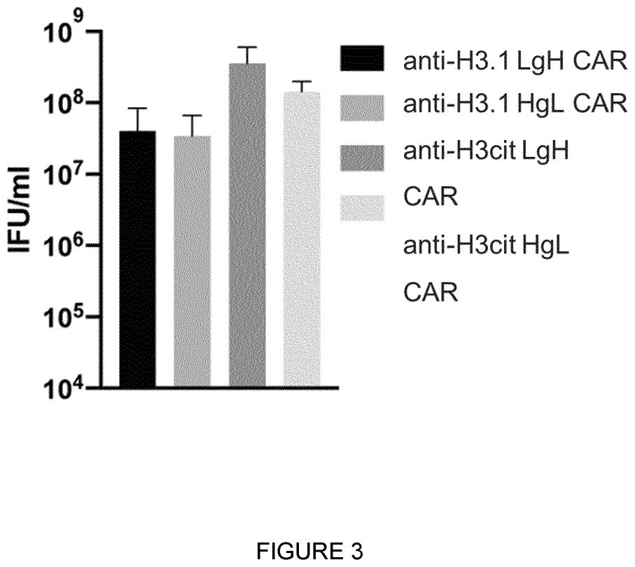

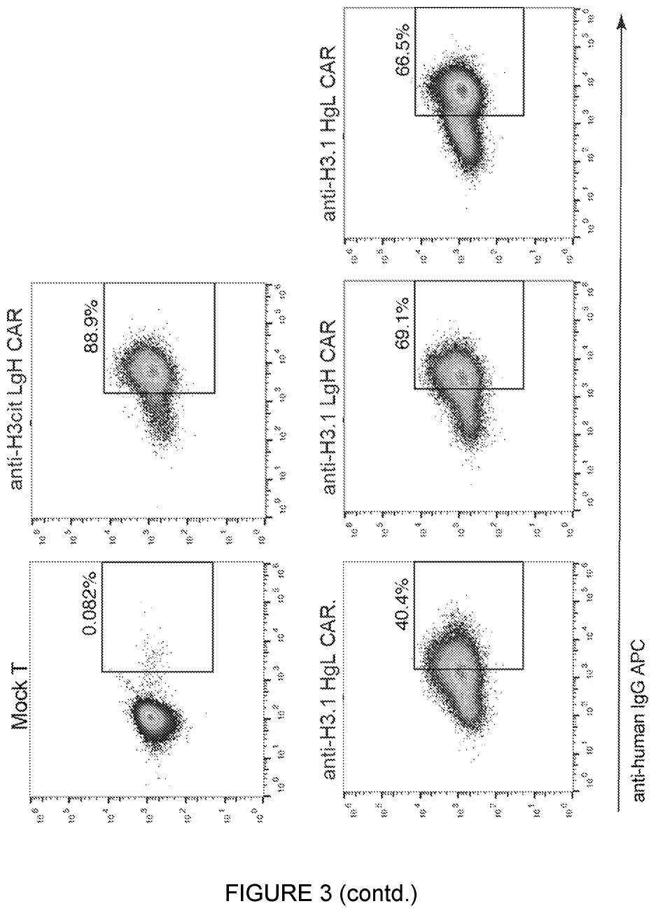

The lentiviral particles were prepared by co-transfection of HEK293T cells with the CAR plasmids and the packaging plasmids pVSVG, pRSV and pMDL. HEK293T cells were cultured in hiDMEM media (High Glucose DMEM (Thermo Fisher Scientific) supplemented with 10% FBS (Thermo Fisher Scientific), 1% non-essential amino acids (Thermo Fisher Scientific), 1% Glutamax (Thermo Fisher Scientific) and 1% sodium pyruvate (Thermo Fisher Scientific)). The day before transfection HEK293T cells were seeded at a concentration of 0.4×10⁶ cells/mL. One hour prior to transfection the media was replaced with fresh pre-warmed hiDMEM. The CAR plasmids were mixed with the packaging plasmids, incubated with Lipofectamine 2000 (Thermo Fisher Scientific) and added to cells. Supernatants containing virus were collected at 48 h post transfection and lentiviruses were concentrated with the Lenti-X Concentrator (Takara). Viral supernatant was centrifuged at 500×g for 10 min and filtered through a 0.45 μm filter to remove debris. Three volumes of supernatant were combined with one volume of Lenti-X™ Concentrator and incubated at 4° C. for 60 min. Samples were centrifuged at 1,500×g for 45 min at 4° C. The supernatant was removed, the pellet was resuspended in 400 μL complete media and stored at −80° C. for long term storage. The titer of lentivirus preparations was determined using Lenti-X p24 ELISAs (Clontech). FIG. 3 shows manufacturing yield of the CAR T lentiviral vectors.

### Example 3: Manufacture of CAR T Cells

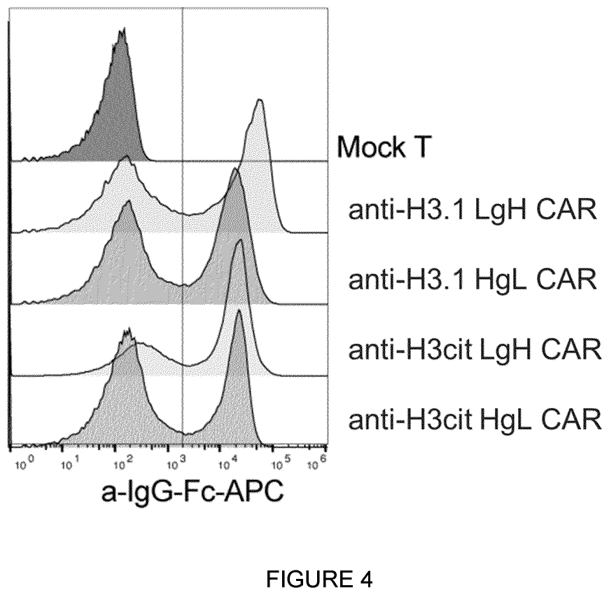

The EasySep Human T Cell Isolation Kit (STEMCELL) was utilized for isolation of T cells from human PBMCs. Human T cells were activated with CD3/CD28 beads at a 1:1 ratio (Life Technologies) in Aim V media (Thermo Fisher Scientific) supplemented with 5% human serum (heat inactivated; Valley Biomedical) and 40 IU/ml recombinant IL-2 (R&D) for 72 hours. Activated T cells were re-suspended to a concentration of 1×10⁶ cells/ml in complete Aim V media supplemented with 10 μg/ml of polybrene (Merck) and concentrated lentiviruses. Plates were centrifuged at 1200×g for 90 minutes at 32° C. and then incubated at 37° C. for 24 hours. After completion of spinfection, CAR-T cells were washed and transferred to a T25 culturing flask (Corning). Culture medium was changed every 2 days and cells were grown in flasks at a density of 1.0×10⁶/ml. The CAR molecules were detected by staining with goat anti-human IgG4 antibody conjugated with APC (Jackson ImmunoResearch). Data was analyzed with FlowJo X10 (FlowJo) and Attune NxT Software (ThermoFisher Scientific). Results are presented in FIG. 4.

The results show that T cells can be efficiently transduced by lentiviral vectors coding the anti-H3.1 LgH, anti-H3.1 HgL, anti-H3cit LgH and anti-H3cit HgL CAR transgenes with efficient follow up expression of CAR molecules by T cells.

### Example 4: Evaluation of Functional Activity of CAR T Cells With Cytotoxic Assay

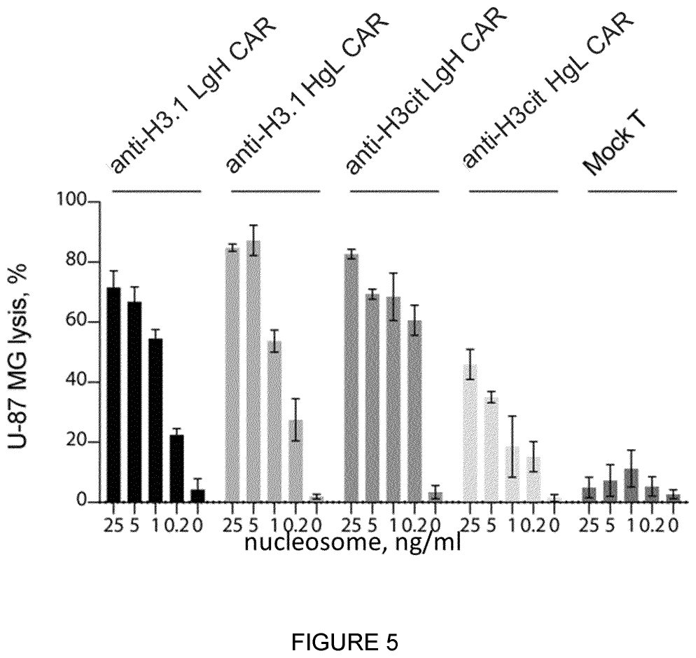

The cytotoxicity and specificity of engineered T cells were evaluated in a standard lactate dehydrogenase release assay (CytoTox 96® Non-Radioactive Cytotoxicity Assay, Promega) following manufacturer's recommendations. The U-87 MG tumor cell line was incubated for 6-12 hours in the presence of anti-H3.1 LgH, anti-H3.1 HgL, anti-H3cit LgH, anti-H3cit HgL CAR T and Mock transduced (CD19 scFv FMC63 clone) T cells plus different quantities of citrullinated mononucleosomes (Active Motif) in a RPMI (Gibco) media supplemented with 40 U/ml of human IL-2 (R&D). To assess the basal level of anti-H3.1 LgH, anti-H3.1 HgL, anti-H3cit LgH and anti-H3cit HgL CAR and Mock T cell cytotoxity, the T cells were incubated in the absence of nucleosomes. The maximum cell lysis was determined by target cell lysis using 10% (vol/vol) cell lysis solution. Target cell cytotoxicity was calculated using the following formula: Cytotoxicity=100×[((CAR-T cell+target cell+nucleosomes)−(CAR-T cell+target cell))/(max target cell lysis−target cells alone)]. The data is presented in FIG. 5.

The results show that anti-H3.1 LgH, anti-H3.1 HgL, anti-H3cit LgH and anti-H3cit HgL CAR T cells are cytotoxic to U-87 MG tumor cell line but only when nucleosomes are present in the culture media. There is a trend for greater cytotoxicity upon increase of nucleosomes quantity in the media.

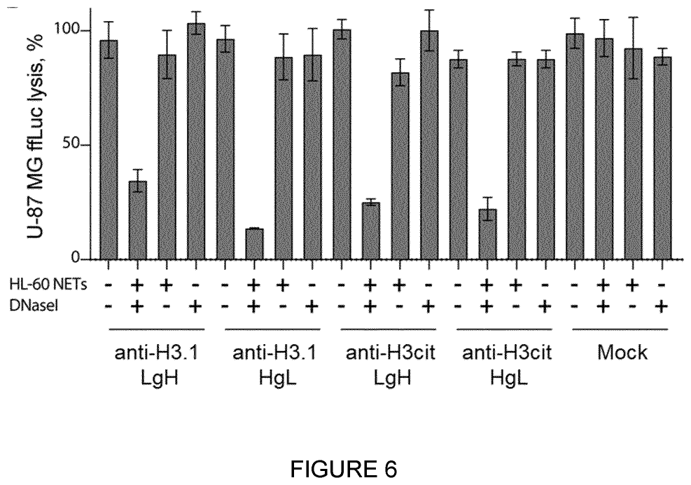

Next, we studied the cytotoxicity of anti-H3.1 LgH, anti-H3.1 HgL, anti-H3cit LgH and anti-H3cit HgL CAR-T cells and Mock scFv control CAR-T cells in the presence of undegraded NETs and DNase I in a luciferase activity assay. The luciferase expressing U-87 MG ffluc cells were mixed with neutrophil extracellular traps (NETs) isolated from HL-60 cells after phorbol 12-myristate 13-acetate (PMA) induction. U-87 MG ffluc cells were incubated for 24 hours with anti-H3.1 LgH, anti-H3.1 HgL, anti-H3cit LgH and anti-H3cit HgL CAR-T cells or Mock T cells in the presence or absence of undegraded NETs and DNase I (10 mg/ml) in a RPMI (Gibco) media supplemented with 40 U/ml of human IL-2 (R&D). The results are presented in FIG. 6.

The results show that anti-H3.1 LgH, anti-H3.1 HgL, anti-H3cit LgH and anti-H3cit HgL CAR T cells are cytotoxic to U-87 MG ffluc tumor cells but only when nucleosomes are released from NETs upon digestion by DnaseI enzyme.

### Example 5: Evaluation of Functional Activity of CAR T Cells With Cytotoxic Assay With Cytokine Release Assay

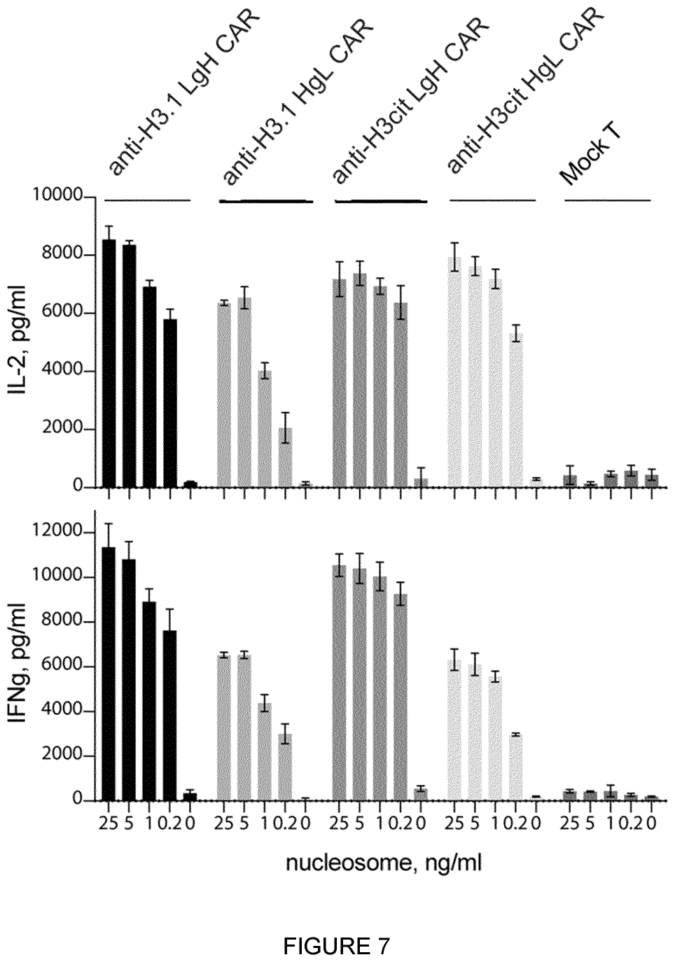

For cytokine release assays, Mock transduced (CD19 scFv FMC63 clone), anti-H3.1 LgH, anti-H3.1 HgL, anti-H3cit LgH and anti-H3cit HgL CAR T cells were incubated with U-87 MG tumor cells together with different quantities of citrullinated mononucleosomes (Active Motif) in complete DMEM (Gibco). After 16 hours of co-incubation the supernatant was separated from cells by centrifugation (4° C., 300 g, 5 min), transferred to a new plate and stored at −20° C. prior use. IL-2 and IFN-γ secretion by human CAR-T cells were analyzed with cytokine-specific ELISA MAX™ Deluxe Set Human IL-2 or IFN-γ kits (BioLegend) according to the manufacturer's instructions. The data is presented in FIG. 7.

The results show that anti-H3.1 LgH, anti-H3.1 HgL, anti-H3cit LgH and anti-H3cit HgL CAR T cells secrete IL-2 and IFN-γ only when nucleosomes are present in the culture media. The cytokine secretion is dose dependent from concentration of nucleosomes in the media.

### Example 6: Model System for Evaluation of Biological Activity of CAR T Cells In Vivo

We are currently exploring in vivo therapeutic efficacy of anti-H3.1 LgH, anti-H3.1 HgL, anti-H3cit LgH and anti-H3cit HgL CAR-T cells in a relevant translational model of cancer. This experiment is set up to determine the anti-tumoral effects of anti-nucleosome CAR T cells alone or in combination with DNase I in male C57BL/6N mice carrying syngeneic colorectal tumor model MC38 hCEA/FFluc. In vivo imaging, flow cytometry, immunohistochemistry (IHC), immunofluorescence (IF) haematoxylin and eosin staining (H&E), biomarker assessment, western blot and the assessment of blood parameters (biochemistry and analytical chemistry) will be performed.

50 male C57BL/6N mice will be subcutaneously implanted with 1×10⁶ cells of the established MC38 hCEA/FFluc syngeneic tumour model cells suspended in 100 μl PBS. Due to limitations in the maximum number of implantations per day, implantation will be carried out on consecutive days and animals will be treated and sampled in several cohorts with an offset of required number of days. The first in vivo imaging will be performed on day 3 PTI when first detectable tumor signals are expected. Animals will be randomized on day 5 PTI into 7 treatment groups of 6 animals each and treated as outlined in the table below. Additional in vivo imaging will be performed at days 10, 17 and 24 PTI. Body weight will be measured three times weekly. Animals will be observed and tumor volumes will be monitored until termination criteria are met (Tumor volume>1000 mm³). Final in vivo imaging will be performed prior to termination of the animal.

### Cancer Cell Injection Conditions

| | | | Number | PBS, | | Unilaterally/ | |
|---|---|---|---|---|---|---|---|
| Cell line | Mouse strain | Gender | of cells | Injection vol. | Route | bilaterally | n= |
| MC38 | C57BL/6N | m | 1 × 10⁶ | 50 μl | s.c. | 1 | 50 |
| hCEA/FFluc | | | | | | | |

### Group Assignment and Therapy Schedule

| | | | NuQ CAR T | | | |
|---|---|---|---|---|---|---|
| | | | dosing days; | | DNase I dosing | |
| Gr | | NuQ CAR T | post | DNase I daily dose* | days post | |
| ID | Compound | dose and route | implantation | [mg/kg/d] and route | implantation | n= |
| 1 | Vehicle | n.a. | n.a. | n.a. | n.a. | 6 |
| 2 | CAR T | 1 × 10⁶ cells i.v. | 10 | n.a. | n.a. | 6 |
| 3 | CAR T | 5 × 10⁶ cells i.v. | 10 | n.a. | n.a. | 6 |
| 4 | CAR T | 1 × 10⁷ cells i.v. | 10 | n.a. | n.a. | 6 |
| 5 | CAR T/D15 | 1 × 10⁶ cells i.v. | 10 | 5 mg/kg; i.v. | 10; 17; 24; 31 | 6 |
| 6 | CAR T/D15 | 5 × 10⁶ cells i.v. | 10 | 10 mg/kg; i.v. | 10; 17; 24; 31 | 6 |
| 7 | CAR T/D15 | 1 × 10⁷ cells i.v. | 10 | 20 mg/kg; i.v. | 10; 17; 24; 31 | 6 |

| | |
|---|---|
| Total no. of mice planned to be randomized: | 42 |

Dosing starts 240 h post implantation. Observation period: max. 4 weeks post last treatment.

In vivo imaging will be carried out during both the pilot study part and the main study on days 3, 10, 17 and 24 PTI. In the pilot study, also a terminal in vivo imaging will be performed while in the main study the terminal imaging will be performed ex vivo on dissected organs. The first imaging of the main study will be performed prior to randomization.

Immunohistochemistry (IHC) and H&E staining: Part of the tumor will be formalin fixed and paraffin embedded in preparation for IHC and H&E staining. Consecutive 5 μm sections will be prepared from each sample to be stained in the following sequence: H&E/Anti-CD45/Anti-CD4/Anti-CD8/Anti-H3cit/Anti-H1.3. Samples will be stained with antibodies each along with suitable isotype controls, all counterstained with hematoxylin. Each of the samples will also separately be stained with Hematoxylin and Eosin (H&E) on a consecutive section. The slides will be digitised using a digital slide scanner. Analysis of stained slides for the percentage of cells stained positive for the above-mentioned antibodies will be performed by an expert pathologist or automated image analysis using by counting the number of stained cells.

Immunofluorescence: Part of the tumor (minimum 100 mm³) will be processed to OCT-embedded blocks for immunofluorescent staining. Upon successful establishment of a staining protocol, MC38 hCEA/FFluc samples will be stained and whole slide scans/regions of interest digitized for downstream analyses using anti-H3cit and anti-Ly6G antibodies.

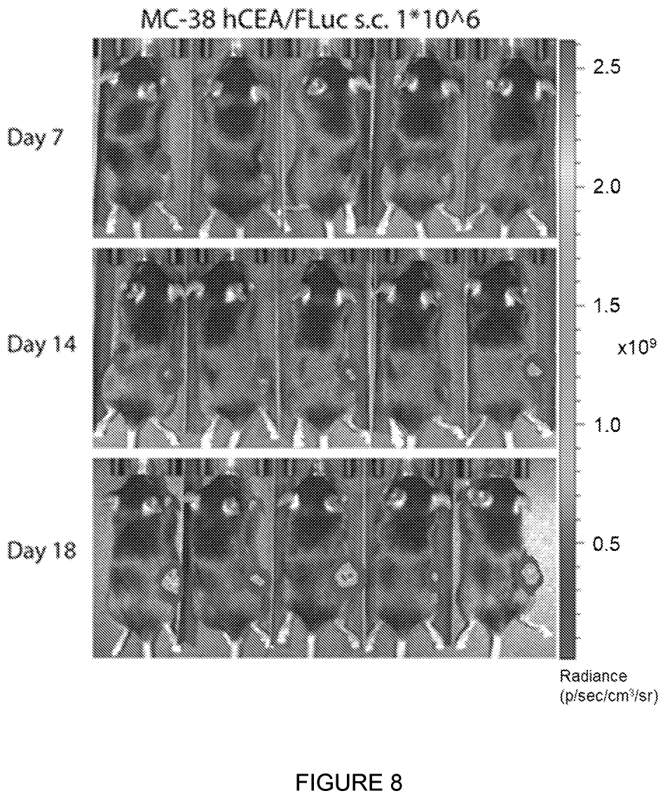

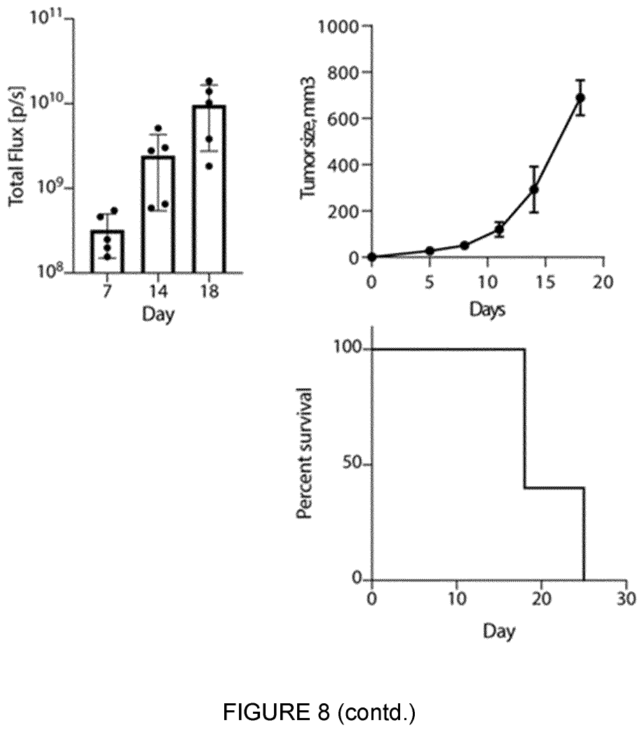

Typical in vivo imaging, tumor growth and survival readouts in MC38 hCEA/Ffluc-C57BL/6N model are presented in FIG. 8.

1. An immune cell, comprising:
a chimeric antigen receptor (CAR) or a T cell receptor (TCR), wherein said CAR or TCR comprises an antigen binding domain capable of specific binding to one or more epitopes associated with neutrophil extracellular traps (NETs).

2. The immune cell of claim 1, wherein the antigen binding domain specifically binds to one or more of a nucleosome, myeloperoxidase, neutrophil elastase, DNA, histone H2A, histone H2B, histone H3, histone H4 and histone H1.

3. The immune cell of claim 1, wherein the epitope is a conformational nucleosome epitope created by the interaction between histones or the interaction between histones and DNA.

4. The immune cell of claim 1, wherein the antigen binding domain specifically binds to an epigenetic feature of a nucleosome, DNA, histone H2A, histone H2B, histone H3, histone H4 or histone H1.

5. The immune cell of claim 4, wherein the epigenetic feature comprises a histone isoform or one or more post-translational histone modifications.

6. The immune cell of claim 5, wherein the one of more post-translational histone modifications comprises at least one modification selected from the group consisting of N-acetylation of serine or alanine; phosphorylation of serine, threonine or tyrosine; N-acylation of lysine; N6-methylation, N6,N6-dimethylation, or N6,N6,N6-trimethylation of lysine; omega-N-methylation, symmetrical-dimethylation or asymmetrical-dimethylation of arginine; citrullination of arginine; ubiquitinylation of lysine; sumoylation of lysine; O-methylation of serine and threonine; phosphorylation of serine, threonine or tyrosine; and ADP-ribosylation of arginine, aspartic acid or glutamic acid.

7. The immune cell of claim 1, additionally comprising a deoxyribonuclease (DNase) enzyme.

8. The immune cell of claim 7, wherein the deoxyribonuclease enzyme is located at or near to the immune cell membrane.

9. The immune cell of claim 7, wherein the deoxyribonuclease enzyme is secreted.

10. The immune cell of claim 9, wherein the deoxyribonuclease enzyme is genetically engineered to contain a targeting domain capable of specific binding to one or more epitopes associated with NETs.

11. The immune cell of claim 7, wherein the deoxyribonuclease enzyme is encoded by a gene therapy vector.

12. A method of producing the immune cell of claim 1, said method comprising introducing into a cell a recombinant adeno-associated virus (rAAV) expression vector or a scaffold matrix attachment region (S/MAR) expression vector comprising a nucleic acid sequence comprising a promoter operably linked to a sequence encoding a CAR or TCR comprising an antigen binding domain capable of specific binding to one or more epitopes associated with NETs.

13. The method of claim 12, wherein the rAAV or S/MAR expression vector additionally comprises a nucleic acid sequence comprising a promoter operably linked to a nucleotide sequence encoding a DNase enzyme.

14-16. (canceled)

17. A method of treating a NETosis related condition in a subject in need thereof, comprising:
administering to the subject an immune cell comprising a CAR or TCR, wherein the CAR or TCR comprises an antigen binding domain capable of specific binding to one or more epitopes associated with NETs.

18. The method of claim 17, wherein the treatment additionally comprises administering a deoxyribonuclease (DNase) enzyme.

19. The method of claim 17, wherein said administering of the immune cell is via fibroin scaffolds.

20. (canceled)

21. The method of claim 17, wherein the NETosis related condition is sepsis, an inflammatory condition or cancer.

22. (canceled)

23. The immune cell of claim 1, wherein the antigen binding domain comprises an scFv.

24. The immune cell of claim 23, wherein the scFv is derived from an anti-histone H3.1 binding antibody or an anti-citrullinated histone H3 (anti-histone H3cit) binding antibody.

25. (canceled)

26. The immune cell of claim 24, wherein the scFv comprises two variable (V) fragments linked via a Ser-Gly linker having the sequence GGGGSGGGGSGGGGS (SEQ ID NO:6).
